(19) United States
(12) Patent Application Publication
(10) Pub. No.: US 2007/0051640 A1
(43) Pub. Date: Mar. 8, 2007

(54) Electrochemical treatment of an aqueous solution

(75) Inventor: Martin Bellamy, Northamptonshire (GB)

Correspondence Address: MORGAN, LEWIS & BOCKIUS LLP, 1701 MARKET STREET, PHILADELPHIA, PA, 19103-2921, US

(73) Assignee: Sterilox Technologies, Inc.

(21) Appl. No.: 11/474,836

(22) Filed: Jun. 26, 2006

Related U.S. Application Data
(63) Continuation-in-part of application No. 10/663,079, filed on Sep. 16, 2003.
Application No. 10/663,079 is a division of application No. 09/633,665, filed on Aug. 7, 2000, now Pat. No. 6,632,347.

(51) Int. Cl. C02F 1/461; C25B 9/00

(52) U.S. Cl. 205/742; 204/242

(57) ABSTRACT

This invention relates to an apparatus and method for producing an output solution having a predetermined level of available free chlorine including two or more parallel production lines. Each production line includes an electrolytic cell, means for passing a saline solution having a substantially constant chloride ion concentration through the cell, means for applying a substantially constant current across the cell, and means for dispensing output solution from the cell.

### CROSS-REFERENCE TO RELATED APPLICATIONS

This application is a continuation-in-part of U.S. patent application Ser. No. 10/663,079, filed Sep. 16, 2003, which in turn is a division of 09/633,665 filed Aug. 7, 2000, now U.S. Pat. No. 6,632,347, and the disclosure of which is incorporated herein by reference.

### BACKGROUND OF THE INVENTION

The present invention relates, among other aspects, to a method of operating an electrochemical cell to produce a biocidal solution and apparatus for producing a biocidal solution by way of the electrolytic treatment of an aqueous chloride solution.

In hospitals it is important to provide appropriate levels of sterility, particularly in operating theatres and other situations where invasive treatments are performed. Surgical instruments and other apparatus must be sterilized or disinfected, depending on their application, before use in order to reduce the risk of bacterial infection. One method of sterilization is the application of heat and pressure in an autoclave. However, this is not suitable for some medical apparatus, such as heat-sensitive endoscopes.

A typical method employed for reprocessing heat sensitive instruments involves the use of chemical biocides, such as glutaraldehyde. This can be unsatisfactory due to improper or incomplete disinfection. Furthermore, exposure to glutaraldehyde fumes can cause asthma and dermatitis in healthcare staff. Also, glutaraldehyde is believed to have relatively low sporicidal activity. Moreover, other disinfectants, such as chlorine dioxide and peracetic acid may suffer from similar handling problems as glutaraldehyde.

For some years, it has been known that electrochemical activation of brine produces a super-oxidized water which is suitable for many applications including general disinfection in medical and veterinary applications and the sterilization of heat-sensitive endoscopes. There has been a recent interest in the use of super-oxidized water as a disinfectant because of its rapid and highly biocidal activity against a wide range of bacteria, fungi, viruses and spores. Also, super-oxidized water is an extremely effective sterilizing cold non-toxic solution which is free from highly toxic chemicals, thereby presenting reduced handling risk.

oxidized water is an extremely effective sterilizing cold non-toxic solution which is free from highly toxic chemicals, thereby presenting reduced handling risk.

GB 2253860 describes the electrochemical treatment of water through an electrolytic cell. Co-axially arranged cylindrical and rod electrodes provide anode and cathode (working and auxiliary) flow chambers which are separated by a porous membrane made of a ceramic based on zirconium oxide.

Water is fed from the bottom to the top of the device through the working chamber. Simultaneously, water having a higher mineral content flows through the auxiliary chamber to a gas-separating chamber. An electric current is passed between the cathode and anode through the water in both chambers and the porous membrane separating the chambers. Water flowing through the auxiliary chamber recirculates to the auxiliary chamber by convection and by the shearing forces applied to the water through the rise of bubbles of gas which are generated on the electrode in the auxiliary chamber. The pressure in the working chamber is higher than that in the auxiliary chamber, and gaseous electrolysis products are vented from the gas-separating chamber by way of a gas-relief valve. A change of working mode from cathodic to anodic water treatment is achieved by changing polarity.

This electrolytic process acts on salts and minerals dissolved in the water, such as metal chlorides, sulphates, carbonates and hydrocarbonates. Where the working chamber includes the cathode, the alkalinity of the water may be increased through the generation of highly soluble metal hydroxides. Alternatively, the electrolytic cell may be switched so that the working chamber includes the anode, in which case the acidity of the water is increased through the generation of a number of stable and unstable acids.

A similar electrolytic cell is described in GB 2274113. This cell includes two coaxial electrodes, separated by an ultra-filtration diaphragm (porous membrane) based on zirconium oxide, thereby defining a pair of coaxial chambers. A current source is connected to the electrodes of a plurality of cells via a switching unit to enable polarity alteration of the electrodes to eliminate deposits on the cathode and to connect the cells electrically either in series or parallel.

WO 98/13304 describes the use of such an electrolytic cell in an apparatus to process a liquid, such as water. A liquid is supplied to the cathode chamber only and part of the output from the cathode (catholyte) is recycled to the input of the anode chamber. This input serves as the total supply to the anode chamber. In situations where not all of the solution output from the cathode chamber is recycled to the input of the anode chamber, a proportion of the output from the cathode chamber is drained to waste, this proportion being measured by a flow meter. A constant-voltage DC supply is applied between the anode and the cathode, and the pH and redox potential of the treated solutions are measured and maintained by controlling flow rates through the cell.

A method and apparatus for producing a sterilizing solution is described in GB 2316090, the subject matter of which is incorporated herein by reference, wherein a supply of softened water is generated by passing water through an ion-exchange water softener. A saturated salt solution, generated by mixing softened water with salt, is passed through an electrolytic cell to produce a sterilizing solution, or used to regenerate the ion-exchange resin in the water softener.

However, all of the systems described above have drawbacks and difficulties. For example, the variable factors, such as the degree of electrolysis in the electrolytic cells, the concentration of dissolved salts and minerals and the flow rates, the fluctuations in electricity supply, ambient temperature and the variability of incoming water supplies present a barrier to ensuring a consistent supply of sterilizing or, more correctly, biocidal solution. Thus in order to ensure delivery of a biocidal solution, the electrochemical systems described all rely upon expert intervention to calibrate the cells at the time of installation and to re-calibrate whenever the chemistry of the water supply changes to any significant degree.

As an illustration, the pH of the solution output from the anode chamber (anolyte) may be regulated by adjusting the flow rate of catholyte drained from the cathode chamber. This results in changes to the anolyte flow rate and consequently in changes to the electrochemistry taking place in the electrolytic cell.

Also, the performance of all the above cells and methods is highly dependent on the alkalinity of the water and aqueous salt solutions being treated. In Europe, for example, the alkalinity of potable water can vary from very low (3-15 ppm CO₃ as CaCO₃) to very high (470 ppm CO₃) from one geographical region to another. This means that a cell which is calibrated to produce a biocidal solution of given composition in a first geographical location may not produce the same biocidal solution in a second location, making re-calibration necessary. This is a time-consuming and laborious task.

Minimizing variation is important to ensure a supply of solution having the required properties, e.g. biocidal activity and pH, especially when thorough sterilization is required to maintain the health of a population.

Furthermore, it is important to be able to control to a fine degree the final composition of any biocidal solution produced, since the solution must have a high enough concentration of, say, available free chlorine (AFC) to be sufficiently biocidal, but not so high as to corrode or otherwise damage any equipment which is being sterilized. A still further disadvantage of the apparatus described in the prior art is that they are prone to a high level of wastage. Up to half of the initial supply of aqueous salt solution may be discarded after being passed through the cathode chamber. This is especially pertinent where resources such as water are limited or costly.

In the Applicant's experience, none of the above systems is suited to providing a wholly reliable or autonomous supply of biocidal solution. As will be readily appreciated, a “sterilizing” solution which does not meet the required level of biocidal efficacy carries a risk of allowing an instrument to spread infection. Moreover, the end user will not be able to detect by visual inspection alone whether the biocidal solution from any one of these systems is within or outside specification.

### SUMMARY OF THE INVENTION

Accordingly, the present invention provides a system which delivers for use a biocidal solution only when it has the desired properties, i.e. it is within specification. In this way, the risk of mistakenly using a solution which is not adequately biocidal can be substantially eliminated. Additionally, increased efficiency is provided by a system which can continue production while part of the system is shut down for cleaning or maintenance.

There is also a need to provide a system which not only is capable of producing a biocidal solution in specification but also on demand. Moreover, there is a further need to provide a system which is able to deliver a biocidal solution in specification, on demand, at or close to where the solution is to be used. In addition, there is a need to provide a system which can operate irrespective of the parameters of the local source of input water. Ultimately, the Applicant has set out to achieve a system which is able to deliver biocidal solution in specification, on demand, continuously, on site, anywhere.

To this end, and as a result of extensive trials and experiments, the Applicant has devised a system which, by virtue of various innovations, ensures that it will continuously deliver biocidal solutions which are within specification. As will become apparent, the Applicant has also devised a system which is able to produce in specification biocidal solution on demand, on site, anywhere.

One embodiment of this invention includes an apparatus for producing an output solution having a predetermined level of available free chlorine including two or more parallel production lines. Each production line includes an electrolytic cell, means for passing a saline solution have a substantially constant chloride ion concentration through the cell, means for applying a substantially constant current across the cell, and means for dispensing output solution from the cell.

Another embodiment of the invention includes a method of electrochemically treating a supply of aqueous salt solution in two or more parallel production lines, where each line can include an electrolytic cell having an anode chamber and a cathode chamber separated by a semi-permeable membrane, the anode and cathode chambers respectively being provided with an anode and a cathode, and each chamber having input and output lines for the solution being treated. For each production line: i) aqueous salt solution is supplied to the anode and cathode chambers by way of their respective input lines, at least the cathode chamber input line being provided with a flow regulator, and output by way of their respective output lines; ii) a substantially constant current is caused to flow between the anode and the cathode; and iii) a proportion of the solution output from the cathode chamber is recirculated to an input line of the anode chamber by way of a recirculation line.

One embodiment of this invention includes a system for electrochemically treating a supply of aqueous salt solution, including two or more parallel production lines. Each production line includes an electrolytic cell having an anode chamber and a cathode chamber separated by a separator, the anode and cathode chambers respectively being provided with an anode or a cathode, and each chamber having input and output lines for the solution to be treated; wherein for each production line: i) the input line to the cathode chamber is provided with a flow regulator; ii) the anode and cathode are connected to a source of substantially constant direct current; and iii) an output line from the cathode chamber is connected to an input line of the anode chamber by way of a recirculation line.

By virtue of the aforementioned features, the Applicant has devised a new system for generating an extremely effective non-toxic, biocidal solution which acts against a wide variety of bacteria, fungi, viruses and spores and is suitable for many applications including disinfection and cold sterilization. In addition, the system can be operated and maintained regardless of location and requires only water, electricity and salt to be put into effect. The system can be operated either continuously or in response to demand and can be adjusted to produce a solution tailored for a particular end use. Moreover, because of the various failsafe means it incorporates, it is virtually impossible for an end user to be provided with a biocidal solution of inadequate efficacy.

In summary, the Applicant has invented a system which is not only adapted always to deliver biocidal solution which falls within the desired specification, but also to deliver such solution continuously on demand, on site, anywhere.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 shows an embodiment of the invention in schematic outline;

FIG. 2 is a detailed flow diagram of the invention as outlined in FIG. 1;

FIG. 3 illustrates a dispenser in accordance with another aspect of the invention;

FIG. 4 shows an electrolytic cell for use in the present invention; and

FIG. 5 is a detailed flow diagram of an embodiment of the invention having two or more parallel production lines.

FIG. 6 is a detailed flow diagram of an embodiment of the invention having an additional apparatus for preparing and storing water for the system.

### DETAILED DESCRIPTION OF THE INVENTION

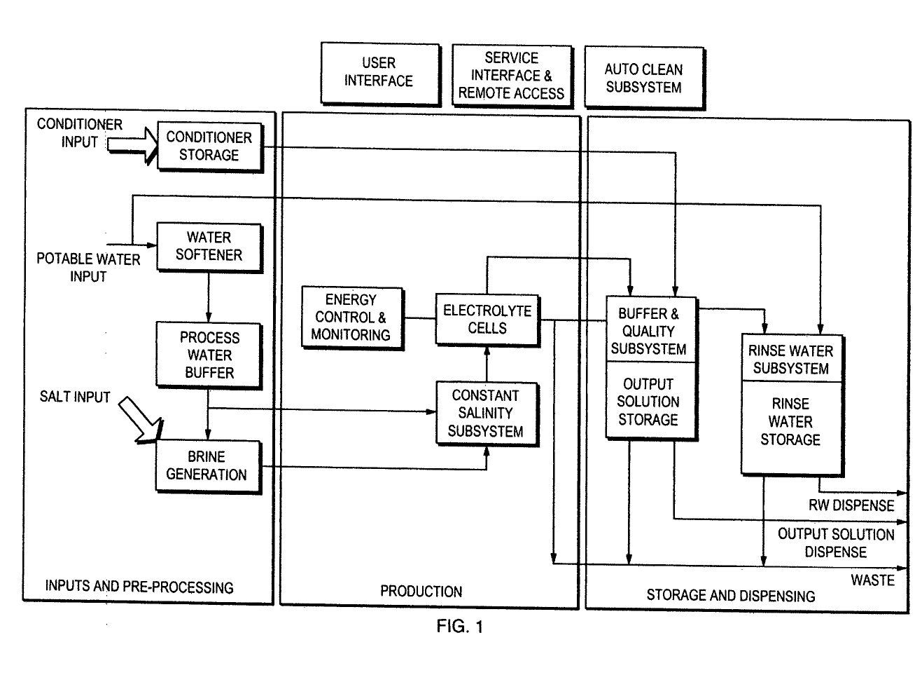

Referring first to FIG. 1, the schematic outline of the invention is broken down into three main processing stages, namely an inputs and pre-processing stage, a production stage and a storage and dispensing stage. While referred to as stages, it will of course be appreciated that the process of the invention may be carried out continuously.

In the first (inputs and pre-processing) stage, there is an input of potable water which, for the purpose of generating saline solution for use in the electrolytic cell, is first passed through a water softener zone where excessive magnesium and calcium ions are removed. The softened water is then passed into a process water buffer zone where it is held until required for use in the production of brine. Potable water input is also passed directly to the storage and dispensing stage for use in the preparation of bacteria-free rinse water, but for this purpose there is no need for the water to be softened prior to use.

The first stage also includes a salt (NaCl) input, usually of vacuum dried crystalline salt which is commercially produced to a consistent standard, to a brine generation zone where a concentrated salt solution is made up from the salt and the softened water obtained via the process water buffer zone.

A further input is provided for additional agents, such as a corrosion inhibitor, used to condition output solution produced by the process. The conditioner is passed to a conditioner storage zone where it is held until required.

Turning to the second (production) stage, this includes a constant salinity subsystem in which a saline solution of substantially constant concentration is produced by dilution of the brine from the brine generation zone with softened water from the process water buffer zone to the desired concentration. The resulting saline solution is passed from the constant salinity subsystem to one or more electrolytic cells, each including cathode and anode chambers (not shown), and across which a substantially constant electric current is applied. The applied electric current is maintained constant via an energy control and monitoring zone.

Catholyte and anolyte are produced from the cathode and anode chambers respectively as a result of the electrochemical treatment of the saline solution in the cells. Anolyte and a portion of catholyte which is not recirculated to the anode chamber are both dealt with in the third (storage and dispensing) stage. In particular, catholyte which is not recirculated is directed to waste and anolyte, otherwise referred to as output solution, is passed to a buffer and quality subsystem. The output solution is tested in the buffer and quality subsystem and, if it fails to meet the quality standards, it is also directed to waste. If the output solution falls within specification, a quantity of conditioner, such as a corrosion inhibitor, is added to it in the buffer subsystem and the output solution is then permitted to pass either into an output solution storage zone from where it is subsequently dispensed for use or into a rinse water subsystem.

Output solution directed to the rinse water subsystem is diluted with potable water from the potable water input and is then passed to a rinse water storage zone from where it is subsequently dispensed.

Provision is also made for discharging output solution from the output solution storage zone and rinse water from the rinse water storage zone to waste.

Information on the various processing stages and the ability to interact with the process is provided by means of a user interface and a service interface. The service interface also provides for remote access to the process, enabling an off-site engineer to obtain information on and make adjustments to the processing in each of the three stages.

There is also provided an autoclean subsystem to permit cleaning of the system, either at regular intervals or whenever convenient.

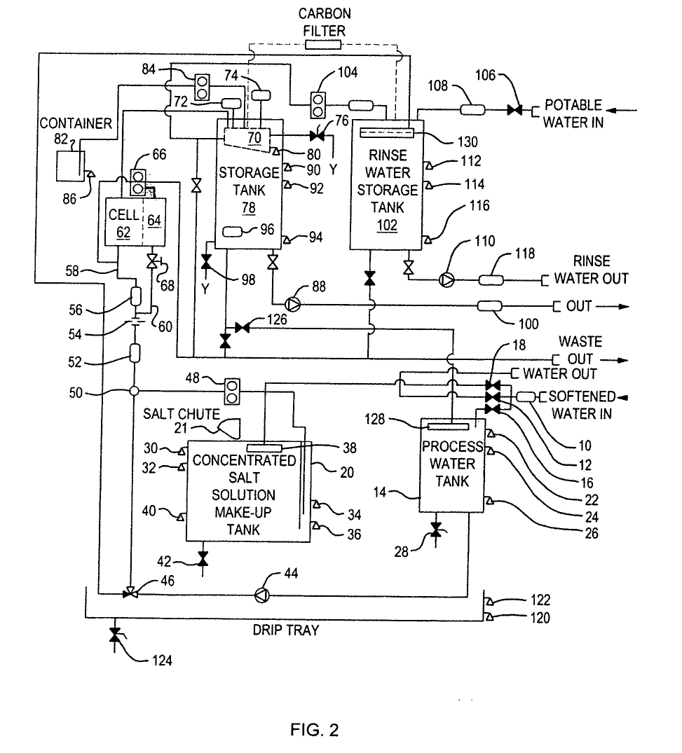

FIG. 2 is a flow diagram or “hydraulic map” showing in more detail the invention already outlined in FIG. 1. Potable water is passed through an external water softener containing a cation exchange resin (not shown) thereby exchanging hardness ions of calcium and magnesium onto the resin and releasing sodium ions into the water.

Incoming softened process water is monitored by sensor 10. Sensor 10 ascertains whether the incoming water is at a temperature within the range under which the process can reasonably operate, namely between about 5 and 35° C. Other parameters such as the incoming water's pressure, softness, alkalinity, pH, conductivity and microbial count can also be monitored by the sensor 10 to establish that it falls within acceptable levels for the process.

If sensor 10 detects that the properties of the incoming softened process water do not fall within acceptable limits required by the specification, the water is diverted through a waste discharge manifold (not shown) to a drain via valve 12. On the other hand, if the incoming softened process water is in specification, it is allowed to flow into internal process water tank 14 through inlet valve 16 or is diverted via inlet valve 18 to concentrated salt make-up tank 20.

Buffer storage for the process water in the event of a temporary interruption in the water supply is provided by the process water tank 14 having a large enough volume. Moreover, tank 14 also has sufficient capacity in order to eliminate pressure fluctuations in the fluid supply to the electrolytic cells. In one embodiment, water tank 14 stores sufficient process water to continue operation for 10 minutes. In another embodiment, water tank 14 stores sufficient process water to continue operation for 15 minutes. In other embodiments, water tank 14 stores sufficient process water to continue operation for 20 minutes, 40 minutes, 60 minutes 90 minutes, or 120 minutes respectively. In another embodiment, water tank 14 stores sufficient process water to continue operation for 180 minutes. Moreover, tank 14 also has sufficient capacity in order to eliminate pressure fluctuations in the fluid supply to the electrolytic cells.

Process water tank 14 includes a plurality of level detectors for monitoring and controlling the process water level in it. In one embodiment, level detector 22 can function as a safety device which is activated when the process water in the tank reaches a predetermined extra high level to stop the charging of the tank with process water and raise an alarm. In another embodiment level detector 24 is activated when the level of liquid in the tank reaches a predetermined high level to stop further inlet water from entering tank 14 by closing valve 16. In another embodiment, water will begin to re-charge tank 14 after a predetermined time has elapsed below the high level. In yet another embodiment, level detector 26 is activated when the process water in tank 14 reaches a low level to prevent production of output solution. In certain embodiments, tank 14 also includes valve 28 which allows liquid to be drained. Furthermore, tank 14 is designed to comply with local regulations, such as the class A air break requirements as required in the United Kingdom by Building Regulations Bylaw 11.

Concentrated salt solution is made-up and stored in concentrated salt solution make-up tank 20. To make up the concentrated salt solution, vacuum dried crystalline salt (BS998: 1990) is added to tank 20 via salt chute 21 having a capacity which is able not only to accommodate a typical salt input of about 6 kg, but to tolerate an amount of overfilling sufficient to keep the system supplied for approximately 1 to 2 days at a normal operation level.

To monitor liquid levels within concentrated salt solution make-up tank 20, level detectors are also provided. Thus, level detector 30 is a safety device which is activated by an extra high level of liquid in tank 20 and acts to close valve 18 to prevent overfilling of tank 20 and to raise an alarm, but will not halt production of output solution. Level detector 32 is activated by a high level of liquid in tank 20 to stop further water filling tank 20 by closing valve 18. Level detector 34 is activated by a low level of liquid in tank 20 and operates to open valve 18 to charge tank 20 with softened water. Low level detector 36 is activated by a very low level of liquid in tank 20 to halt production of output solution and to raise an alarm.

In certain embodiments, softened water is fed through valve 18 and automatically fills tank 20 through spray-bar 38 until high level switch 32 is activated. Salt in tank 20 dissolves in the water to produce a concentrated salt solution with the level of salt reducing as more salt is dissolved.

Further level detector 40, this time for the salt, is located towards the bottom of tank 20. Salt level detector 40 is activated when the amount of salt in tank 20 is depleted such that it is approaching a level insufficient to produce a concentrated salt solution. On activation, an alarm is raised which alerts an operator that more salt is required. The request to add salt is displayed on the user interface (FIG. 1) and replenishment of the salt supply in tank 20 may be carried out manually by an operator or automatically through a control system. The user interface is operative to display a suitable message when sufficient salt has been added.

Finally, tank 20 also includes a manual drain valve.

Concentrated salt solution from salt make-up tank 20 is diluted with process water from process water tank 14 to produce a saline solution of substantially constant chloride ion concentration. In more detail, process water is continuously pumped by process water pump 44 through valve 46 towards an electrolytic cell pack and concentrated salt solution is pulse fed into the flow of process water via an adjustable speed peristaltic pump 48. The pulses of concentrated salt solution are dispersed into the substantially continuous stream of process water through a perforated tube 50 thereby evening out the pulses to produce a flow of saline solution of uniform concentration.

Preferably dispenser 50 is substantially elongate, for example in the form of a length of tube having an external diameter less than the internal diameter of the conduit, which itself may comprise a tube, and has a closed end and an open, feed end. For maximum effect, the apertures are preferably arranged substantially evenly both longitudinally and circumferentially of dispenser 50. Conveniently the apertures comprise perforations and their size may be varied.

The flow rate of the resulting saline solution as it flows towards the cell pack is monitored by flow meter 52 and if necessary is modulated by a flow regulator in the form of orifice plate 54. The flow rate is changed simply by changing the size of the orifice in the plate. Different orifice plates may be chosen to suit site conditions.

Applicant has found that a saline solution diluted to a concentration of less than 1% w/vol, more preferably in the region of 0.3%, is particularly suitable. The preferred concentration will however be determined according to a number of factors specific to the electrolytic cell being used and the type of output solution desired.

The final concentration of the mixed saline solution will be determined by the volume of dispenser 50, the pulsing rate of concentrated salt solution into dispenser 50 and the flow rate of the water diluent. For example, the Applicant has calculated that, to produce a 0.3% saline solution from a concentrated salt solution of about 12% w/vol, dispenser 50 should have a length in excess of about 0.19 m. Ideally, the perforations in dispenser 50 have an inner diameter of approximately 1 mm, and that about ten perforations are sufficient for this application.

In a typical system practicing the method of the invention, the concentrated salt solution is preferably pulsed at a rate of between about 1 to 5 liters per hour and the water diluent is supplied at a rate of between about 150 to 250 liters per hour to achieve the target chloride concentration from dispenser 50.

It is preferred if the concentrated salt solution is pulse fed into a flow of diluent water, for example by means such as a peristaltic pump. In this way, each pulse is directed to deliver a known quantity of concentrated salt solution. Accordingly, as the concentrated salt solution becomes more dilute, for example as the supply of salt is depleted, the pulsing rate of the concentrated salt solution into the water flow is increased.

The Applicant has found that benefits are achieved by periodically allowing the concentrated salt solution to become increasingly dilute. By such means, deposits of crystalline salt in the apparatus in which the concentrated salt solution is prepared are reduced.

Prior to entering the cell pack, the concentration of chloride ions in the saline solution is checked by means of conductivity sensor 56. If the conductivity measurement indicates that the chloride ion concentration has fallen below the desired level or has risen above it, the pulsing rate of peristaltic pump 48 is increased or decreased respectively to alter the amount of chloride ions being dispersed into the process water through perforated tube 50 thereby compensating for the fall or rise in chloride ion concentration. The size of the aperture in orifice plate 54 is also adjusted to regulate the flow of chloride ions into the cell pack. Adjustment of the pulsing rate and the flow rate together provide a fine tuning means to ensure that the cell pack is supplied with a constant chloride ion throughput.

On the other hand, if the conductivity of the saline solution as measured by conductivity sensor 56 falls outside a predetermined range such that it is not possible to adjust the pulsing rate and/or flow rate to bring the conductivity within the required range, and hence make it virtually impossible for the cell pack to produce output solution having the desired level of available free chlorine, an alarm is raised and the flow of saline solution to the cells is ceased pending rectification of the problem.

In some embodiments, if the saline solution already provides or can be adjusted to provide the requisite throughput of chloride ions, it can be split into two streams 58, 60 before being fed through the cell pack. In other embodiments, the solution can continue to flow as one stream, traveling through the cathode chamber first and through the anode chamber second. Typically the cell pack consists of eight electrochemical cells, with two sets of four cells connected hydraulically in parallel. For simplicity, only one cell is illustrated. However, the number of cells in the cell pack is determined by the output volume required from the particular system. Each cell has anode chamber 62 and cathode chamber 64 and the flow of saline solution is split such that the greater portion is fed to anode chamber 62 and the lesser portion is fed to cathode chamber 64. In this embodiment, approximately 90% of the saline solution is passed through the anode chamber(s) with the remainder passed through the cathode chamber(s). The flow rate of saline solution through the cathode chamber is much lower than for the anode chamber and the pressure in the cathode chamber is also lower.

It will be generally understood that the function of a separator in the cell is to isolate the solution in one chamber from the solution in the other chamber while allowing the migration of selected ions between the chambers and the term “separator” as used herein should be construed accordingly. Semi-permeable diaphragms and ion-selective membranes are the most common forms of known separators.

In an electrochemical reaction, it is known that the rate of reaction is generally directly proportional to current within certain limits of the current. Therefore, the current (and thus the rate of oxidation of chloride to chlorine) and flow of chloride through the cell may be set appropriately to produce an output solution having the predetermined level of available free chlorine. The desired current will depend not only on the type of cell being used, for example, the material from which the electrodes are made and the various rare metals used to provide active coatings on the electrodes, but also the size of cell, for example, for a cell having an anode surface area of approximately 100 cm², an applied current between cathode and anode of 8 Amps is particularly suitable.

In general, the voltage will change as the resistance of the electrolytic cell changes, for example, through deposition of scale in the separator. Accordingly, if the voltage, but not the current, is kept constant, the resistance in the cell will increase as the cell is used. In accordance with Ohms Law, the current will drop and therefore the concentration of available free chlorine in the output solution will fall. This will result in an output solution which may not have sufficient available free chlorine to enable it to act as a biocide.

However, the Applicant has appreciated that under conditions of constant current, the voltage across the electrolytic cell can be monitored usefully to provide an indicator of other parameters, such as the performance of the apparatus used to carry out the method.

As the saline solution flows through the electrolytic cells, a fixed current of between 7-9 amps (typically 8A) is applied to each cell causing electrolysis of the saline solution thereby generating available free chlorine in the resulting anolyte, elsewhere generally referred to as the output solution. In order In embodiments where the flow is split between the anode chamber and cathode chamber, the pH of the output solution can be at least partially controlled to produce output solution at a relatively neutral pH, namely between 5 and 7, by dosing a portion of the catholyte to inlet stream 58 for anode chambers 62. The catholyte can be dosed to inlet stream 58 by adjustable peristaltic pump 66 and the dosing rate can be increased or decreased to achieve the target pH. In this way, the system is also adapted to cope with varying alkalinity of the input potable water. The remaining catholyte which is not dosed into input stream 58 for anode chambers 62 can be directed to waste, if necessary diluting it prior to disposal.

Since the flow rate of the saline solution into cathode chamber 64 also has an influence on the pH of the output solution, flow regulator 68 is provided to control the flow of saline entering the chamber. Flow regulator 68 can be manually adjusted if there is a variation in input water quality. Output solution is fed from the outlet of anode chambers 62 of the cell pack into intermediate weir tank 70.

The level of available free chlorine will be set according to the biocidal properties which are required to be imparted to the output solution. The output solution will preferably be required to act as a biocide against a wide range of bacteria, fungi, viruses and spores. An available free chlorine content of about 3 ppm to 300 ppm will generally provide biocidal properties for most envisaged applications. It will however be appreciated that biocidal efficacy is also dependant on pH and therefore that an appropriate balance must be achieved between pH and AFC in order to provide the desired level of bio-compatibility and materials compatibility. For example, the Applicant has found that a level of available free chlorine of approximately 100-250 ppm at a pH of between about 5 and 7 is particularly suitable for the application of reprocessing heat sensitive medical instruments. Other applications, such as its use in non-medical environments, for example as in the processing of poultry and fish and general agricultural and petrochemical uses, the breaking down of bacterial biofilm and water treatment, may demand different levels of available free chlorine.

The pH and redox potential of the output solution in weir tank 70 are measured by pH meter 72 and redox probe 74 respectively. If the pH and redox potential do not fall within the desired parameters, valve 76 is opened and the contents of weir tank 70 are drained to waste. The contents of tank 70 are drained to waste in any event if they have remained in the tank for about three hours. pH meter 72 is linked to pump 66 to adjust the level of catholyte dosed to anode chambers 62 thereby enabling the pH of the output solution to be adjusted to bring the output solution within the desired pH range. If the pH and redox potential of the output solution are determined to fall within the desired parameters, confirming that the output solution has the necessary biocidal efficacy, valve 76 is kept closed and the output solution is allowed to fill weir tank 70 until it reaches a level where it floods over into storage tank 78. Weir tank 70 includes level detector 80 for monitoring when the level of output solution in the tank falls to a predetermined low level. When low level detector 80 is activated, the production of sterile rinse water is stopped.

Provided pH meter 72 and redox probe 74 confirm that the output solution has the desired parameters, a corrosion inhibitor, such as a mixture of sodium hexametaphosphate and sodium molybdate, is dosed as a solution from storage container 82 into the output solution in weir tank 70 by peristaltic pump 84. Sensor 86 is mounted in the storage container to monitor low levels of inhibitor and trigger an alert mechanism which alerts the system that there is a need for inhibitor to be supplied to storage container 82.

In specification output solution spills from weir tank 70 into storage tank 78 where it remains until a demand for it is received. For example, when it is required for a cycle of a washer-disinfector machine, the system receives a demand signal from a washing machine interface control module triggering operation of dispensing pump 88. Typically, dispensing pump 88 is rated so that it can supply output solution to three washing machine vessels of 25 litre capacity in 180 seconds (1500 liters per hour, 3 bar line pressure). The capacity of storage tank 78 is therefore such that it too can fulfill the volume requirement.

Storage tank 78 includes various level detectors for monitoring liquid levels in the tank. Level detector 90 is activated by an extra high level of output solution within the tank, raising an alarm and stopping production. Level detector 92 is activated before detector 90 as the volume of output solution rises in storage tank 78 and simply stops production. As the output solution is dispensed and after a period of time below the level of detector 92, production of output solution is recommenced. Low level detector 94 is activated when the level of the output solution falls to a low level, raising an alarm and preventing further dispensing to the machine.

pH probe 96 for monitoring the pH of the output solution is provided within storage tank 78 so that if the pH of the output solution drops out of specification, it is routed to waste by valve 98 located on the outlet of storage tank 78. In addition, if the output solution has been stored for 24 hours, it is similarly routed to waste. In this way, output solution which is out of specification is never dispensed. In order to monitor the flowrate and amount of output solution dispensed from storage tank 78, flow meter 100 is linked to ‘no flow’ and leak detection routines within a user/service interface to alert the system, for example, that discharge valve 98 is closed during a requested discharge, or that an unrequested discharge is occurring.

The Applicant believes that, to ensure the biocidal efficacy of the bacteria-free rinse water, the output solution used to make up the rinse water is preferably not more than about three hours old. In accordance with various failsafe provisions in the preferred method of the invention, any output solution which is detected to fall outside the required specification is generally discharged to waste regardless of its age. However, for the purposes of bacteria-free rinse water, even “in specification” output solution will not be used to generate bacteria-free rinse water if it is more than the desired maximum age.

Since the output solution held in weir tank 70 is never more than three hours old, it is used to produce bacteria-free rinse water. Fresh output solution is dosed at a predetermined rate from weir tank 70 to rinse water storage tank 102 via peristaltic pump 104. In certain embodiments, filtered potable water flows into tank 102 through valve 106 where it is mixed with and dilutes the output solution to a concentration of about 1-15%. In one embodiment, the output solution is diluted to a concentration of about 1-3%. In another embodiment, the output solution is diluted to a concentration of about 2%. In certain embodiments, if the local water supply is of poor quality, a higher concentration of output solution in the rinse water, for example, about a 5% solution, is preferred. Accordingly, the dosing rate of pump 104 is determined by the incoming potable water supply and is monitored by flowmeter 108. Both potable water and output solution are added to rinse water storage tank 102 simultaneously and a minimum standing time of two minutes is always allowed before dispensing the resulting mix. This ensures sufficient contact time for the output solution to diffuse in and activate the potable water. Rinse water is stored in rinse water storage tank 102 until it is required by, for example, an endoscope washing machine. Dispensing pump 110 is activated on receipt of a demand signal from a washing machine interface control module. As with dispensing pump 88, dispensing pump 110 is similarly rated to meet the demand of filling three washing machine vessels of 25 liters capacity in 180 seconds (1500 liters per hour, 3 bar line pressure) and the capacity of the rinse water storage tank 102 is also dictated by this typical demand scenario.

In certain embodiments, rinse water tank 102 is provided with a plurality of level detectors to monitor levels of rinse water. Level detector 112 is activated when there is an extra high level of rinse water in tank 102, alerting the system and stopping any further production of rinse water. Level detector 114 monitors high rinse water level in tank 102 and when activated stop rinse water production. After a predetermined period of time has elapsed and when the rinse water level has fallen, high rinse water level detector 114 is deactivated and the production of rinse water is recommenced. When there is only a low level of rinse water in tank 102, level detector 116 is activated raising an alarm and preventing further rinse water from being dispensed.

The flowrate and total rinse water dispensed is monitored by flowmeter 118, which also is used in ‘no flow’ and leak detection routines linked to the user/service interface (FIG. 1). By automatic monitoring of liquid levels in weir tank 70, storage tank 78 and rinse water tank 102, and by discharging the output solution and rinse water periodically, the system is able to self-adjust to allow it to meet demand at all times. Gases generated by the electrolytic reaction in the cell pack, mainly hydrogen and chlorine, are vented through a carbon filter located above weir tank 70 and rinse water tank 102 to reduce the quantity of chlorine which escapes.

The system also includes a drip-tray provided with leak detection means in communication with the user/service interface (FIG. 1). The drip tray is a shallow vessel housing two level detectors 120, 122, one being a low level detector and the other an extra high level detector. Low level indicator 120 is activated by any small leak within the machine and activates an alarm when the liquid level rises above the detector, but does not halt the production process in any way. However, extra high level detector 122 activates an alarm and halts the production and dispensing of output solution. Manual valve 124 is provided at the base of the drip tray to allow drainage of the tray.

To maintain the system properly, it is necessary to sterilize the storage tanks and discharge lines on a regular, typically daily, basis. Output solution having the desired biocidal properties as confirmed by its measured parameters and age is flushed through the filters, tanks and pipework to eliminate bacterial growth in these areas. In particular, before the cleaning cycle is commenced, output solution tank 78 is replenished to a high level as detected by detector 92 ensuring that sufficient output solution is available for the cycle, and the pH and redox potential of the output water are confirmed as being within specification by pH probe 72 and redox probe 74. The pH and redox potential will change during the cleaning process and need not be monitored once the cleaning process has commenced. On the other hand, rinse water tank 102 and process water tank 14 are drained to low level prior to commencing the cleaning cycle.

Output solution from storage tank 76 is routed via valve 126 to fill process water tank 14 via spray bar 128. Spray bar 128 causes the output solution to be sprayed onto the tank walls throughout the filling process. Once process water tank 14 is full to the predetermined level, the output solution is pumped by pump 44 through the cell pack into weir tank 70. The output solution is then drained to waste via valve 76.

When the “cleaning” output solution reaches a low level in process water tank 14 as detected by level detector 26, tank 14 is re-filled with output solution via valve 126. Output solution is then pumped by pump 44 from process water tank 14 and valve 46 is opened to divert the output solution to rinse water tank 102 via spray bar 130. When rinse water tank 102 is filled, the tank is held full for about five minutes in anticipation of a demand to flush the rinse water line. If no signal is received, rinse water tank 102 is allowed to drain along with process water tank 14 and storage tank 76.

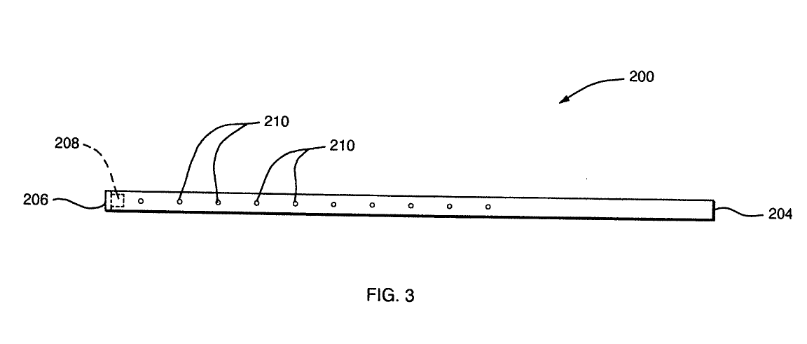

FIG. 3 shows a dispenser 200 for uniformly dispersing two miscible liquids. Dispenser 200 is in the form of elongate tube 202 having open first end 204 and second end 206 closed by end cap 208. Tube 202 is provided with a row of perforations 210 substantially along its length. In use in the method of the invention, open first end 204 of dispenser 200 is fixed to the end of the feed line for the concentrated salt solution which is pulse fed from make up tank 20 (FIG. 2) by peristaltic pump 48. Dispenser 200 is located in and aligned with the flow path of process water which is continuously pumped from process water tank 14 by pump 44. As the pulses of concentrated salt solution arrive in dispenser 200, the solution is forced out through perforations 210 into the process water flow. The resulting saline solution is of substantially homogeneous concentration by virtue of the mixing pattern achieved by dispenser 200.

The dilution of the saturated salt solution is determined by the length of dispenser 200, or rather the length over which the perforations are provided, the pulse rate of the saturated salt solution and the velocity of the process water.

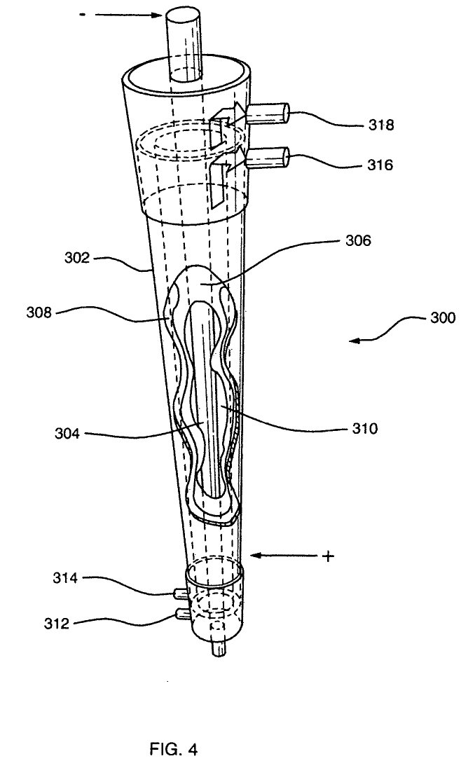

FIG. 4 shows electrolytic cell 300 as used in the present invention. Cell 300 comprises co-axial cylindrical and rod electrodes 302, 304 respectively, separated by semi-permeable ceramic membrane 306 co-axially mounted between the electrodes thus splitting the space between the electrodes to form two chambers 308, 310. Cylindrical electrode 302 forming the anode is typically made from commercially pure titanium coated with an electrocatalytic (active) coating suitable for the evolution of chlorine from a chloride solution. Rod electrode 304 forming the cathode is made from titanium and machined from an 8 mm stock bar to a uniform cross-section over its effective length, which is typically about 210 mm.+−.0.5 mm. Semi-permeable ceramic membrane 306 forming a separator and creating anode and cathode chambers 308 and 310 is composed of aluminium oxide (80%), zirconium oxide (18.5%) and yttrium oxide (1.5%), and has a porosity of about 50-70%, a pore size of 0.3 to 0.5 microns and a wall thickness of 0.5 mm+0.3 mm/−0.1 mm. The ceramic of membrane 306 is more fully disclosed in the specification of patent application number GB 9914396.8, the subject matter of which is incorporated herein by reference.

Cell 300 is provided with entry passages 312, 314 to permit the saline solution to enter cell 300 and flow upwards through anode and cathode chambers 308 and 310 and is discharged as anolyte and catholyte through exit passages 316, 318 respectively. The anolyte containing available free chlorine constitutes the output solution.

As previously described, in order to provide a useful amount of output solution within a reasonable period of time, a group of cells are connected together to form a cell pack. For example, a cell pack comprising eight cells connected together in parallel hydraulically and in series electrically is capable of generating about 200 liters/hour of output solution.

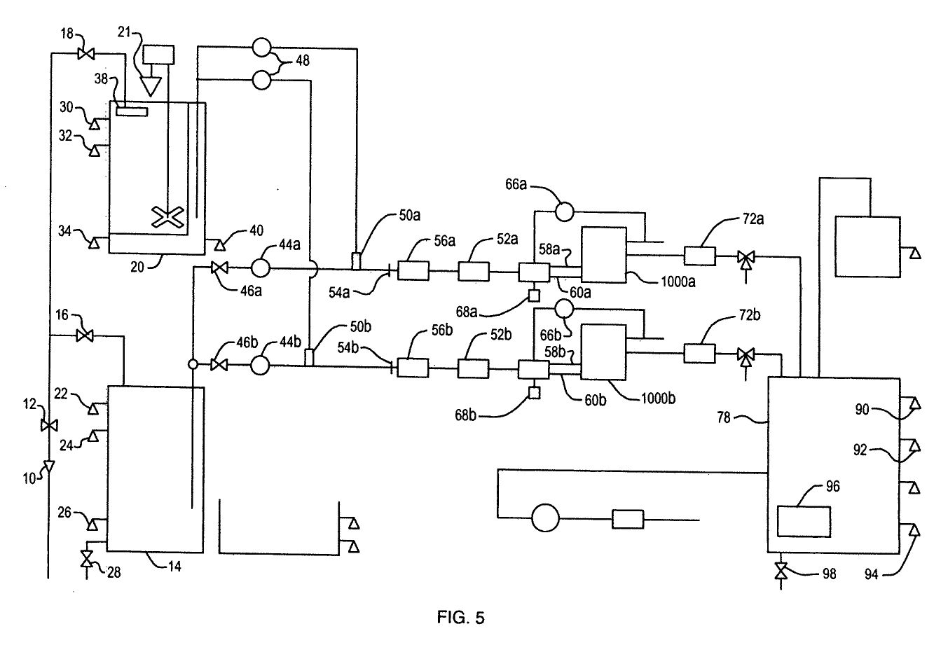

FIG. 5 is a flow diagram or “hydraulic map” showing in more detail an embodiment of the invention including two or more parallel production lines which are fed from the same or different production water and salt tank(s), and which outlet to the same storage device (e.g., tank). In one embodiment, potable water is passed through an external water softener containing a cation exchange resin (not shown) thereby exchanging hardness ions of calcium and magnesium onto the resin and releasing sodium ions into the water.

With reference to FIG. 5, incoming softened process water may be monitored by optional sensor 10. Sensor 10 ascertains whether the incoming water is at a temperature within the range under which the process can reasonably operate, namely between about 5 and 35° C. Other parameters such as the incoming water's pressure, softness, alkalinity, pH, conductivity and microbial count can also be monitored by additional optional sensors (not shown) to establish that the parameters fall within acceptable levels for the process. A feedback loop may be employed to adjust the parameters if the sensors detect that the parameters are outside of the acceptable levels for the process. Unless otherwise specified, the components labeled in FIG. 5 and process specifications are the same as set forth above.

In certain embodiments, softened water can be fed through valve 18 and automatically fills tank 20 through spray-bar 38 until high level switch 32 is activated. In some embodiments, softened water can have a pH of about 4.0-10.0. In some embodiments, softened water can have a pH of 5.5-8.0. The generation of softened water for use in the present invention is described further below.

With respect to FIG. 5, concentrated salt solution from the salt make-up tank 20 is diluted with process water from process water tank 14 to produce a saline solution of substantially constant chloride ion concentration. In more detail, process water is continuously pumped from process water tank 14 through a line which splits into two or more individual lines. Each individual line has at least one process water pump 44a and/or b which continuously pumps the process water through optional valves 46a and/or b towards electrolytic cell pack 1000a and/or b. Pump 44a and/or b can be any suitable pump, including a standard pump or an oscillating pump. Each line receives concentrated salt solution from an individual salt concentrate line from salt tank 20. In certain embodiments, the salt concentrate is pumped from salt tank 20 and is pulse fed into the process water in each of the two or more individual lines via adjustable pumps 48a and/or b on the individual salt concentrate lines. In certain embodiments, pumps 48a and/or b can be peristaltic. The pulses of concentrated salt solution are dispersed into the substantially continuous stream of process water in each individual line through injection quill or perforated tube 50a and/or b thereby evening out the pulses to produce a flow of saline solution of uniform concentration. In some embodiments, the initial conductivity of the process water can be adjusted by the injection of the concentrated salt solution into the process water. In some embodiments, process water can be adjusted to have a conductivity at 20° C. of up to 900 μS/cm. In some embodiments, process water can be adjusted to have a conductivity at 20° C. of up to 800 μS/cm. Salt in tank 20 dissolves in the water to produce a concentrated salt solution with the level of salt reducing as more salt is dissolved

The flow rate of the resulting saline solution as it flows towards cell pack 1000a and/or b in each individual line can be monitored by flow meter 52a and/or b and if necessary is modulated by a flow regulator in the form of orifice plate 54a and/or b. In certain embodiments, the flow rate is changed simply by changing the size of the orifice in the plate. Different orifice plates may be chosen to suit site conditions. In another embodiment, orifice plate 54 and/or b can be removed and the flow can be directly adjusted by pumps 44 and/or b.

Prior to entering cell pack 1000a and/or b, the concentration of chloride ions in the saline solution is checked in each individual line by means of a temperature compensated conductivity sensor 56a and/or b. If the conductivity measurement indicates that the chloride ion concentration in that line has fallen below the desired level or has risen above it, the pulsing rate of corresponding pump 48a and/or b is increased or decreased respectively to alter the amount of chloride ions being dispersed into the process water in the individual line through corresponding perforated tube 50a and/or b thereby compensating for the fall or rise in chloride ion concentration. In certain embodiments, the size of the aperture in optional orifice plate 54a and/or b can also be adjusted to regulate the flow of chloride ions into corresponding cell pack 1000a and/or b. Adjustment of the pulsing rate and the flow rate together can function as a fine tuning means to ensure that cell pack 1000a and/or b is supplied with a constant chloride ion throughput.

Again with reference to FIG. 5, in certain embodiments, if the conductivity of the saline solution in the individual line as measured by conductivity sensor 56a and/or b, falls outside a predetermined range such that it is not possible to adjust the pulsing rate and/or flow rate to bring the conductivity within the required range, and hence make it virtually impossible for cell pack 1000a and/or b to produce output solution having the desired level of available free chlorine, an alarm can be raised and the flow of saline solution to cell pack 1000a and/or b in the corresponding individual line can be ceased pending rectification of the problem. Each individual line can be operated and adjusted separately, independent of the function of any other lines.

In certain embodiments, if the saline solution already provides or can be adjusted to provide the requisite throughput of chloride ions, the individual line can be further split into two streams 58a and/or b, and 60a and/or b before being fed through cell pack 1000a and/or b. In certain embodiments, each cell pack 1000a and/or b includes one or more electrochemical cells connected hydraulically in parallel. In one embodiment, each cell pack contains 4, 6, 8, or more cells, each connected hydraulically in parallel. In one embodiment, cell packs can be connected in series on an individual line. In another embodiment, a cell pack on an individual line can be connected in parallel to a cell pack on another individual line. In another embodiment, cell packs can be connected in series on an individual line, and in parallel with one or more cell pack, on another individual line. As with FIG. 2, for simplicity, only one cell for each line is illustrated. However, the number of cells in the cell pack 1000a and/or b can be determined by the output volume required from the particular system. Each cell of cell pack 1000a and/or b has anode chamber 62 and cathode chamber 64.

In certain embodiments, the flow of saline solution can be split into streams 58a and/or b and 60a and/or b, as described above, such that the greater portion is fed to anode chamber 62 and the lesser portion is fed to cathode chamber 64 of each cell of cell pack 1000a and/or b. In one embodiment, approximately 50-99% of the saline solution is passed through the anode chamber(s) with the remainder passed through the cathode chamber(s). In one embodiment, approximately 80-95% of the saline solution is passed through the anode chamber(s) with the remainder passed through the cathode chamber(s). In one embodiment, approximately 85-92% of the saline solution is passed through the anode chamber(s) with the remainder passed through the cathode chamber(s). In yet another embodiment, approximately 90% of the saline solution is passed through the anode chamber(s) with the remainder passed through the cathode chamber(s). In certain embodiments, the flow rate of saline solution through the cathode chamber is much lower than for the anode chamber and the pressure in the cathode chamber is also correspondingly lower. In certain embodiments, the control can be automated to adjust the percentage of solution fed to the anode chamber.

With respect to FIG. 5, as the saline solution flows through electrolytic cells 1000a and/or b, a current can be applied to each cell 1000a and/or b causing electrolysis of the saline solution thereby generating available free chlorine in the resulting anolyte, elsewhere generally referred to as the output solution. In one embodiment, the current is fixed. In one embodiment, the current is about 5-15 amps. In another embodiment, the current is about 6-13 amps. In yet another embodiment, the current is about 7-10 amps. In yet another embodiment, the current is about 8 amps. In certain embodiments, the pH of the output solution can be at least partially controlled by dosing a portion of the catholyte to inlet stream 58a and/or b for anode chambers 62, in order to produce output solution at a relatively neutral pH, namely between 5 and 7. In certain embodiments, the catholyte can be dosed to inlet stream 58a and/or b by adjustable pump 66a and/or b and the dosing rate can be increased or decreased to achieve the target pH. In one embodiment pump 66a and/or b can be peristaltic. As noted above, the system can also adapt to cope with varying alkalinity of the input potable water. As also described above, any remaining catholyte which is not dosed into input stream 58a and/or b for anode chambers 62 can be directed to waste, if necessary diluting it prior to disposal.

Since the flow rate of the saline solution into cathode chamber 64 of each cell of cell pack 1000a and/or b can also have an influence on the pH of the output solution, in certain embodiments flow regulator 68a and/or b can be used to control the flow of saline entering the chamber for each individual line, as detailed above with respect to FIG. 2. Flow regulator 68a and/or b can be manually or automatically adjusted if there is a variation in input water quality. Output solution can be fed from the outlet of anode chambers 62 of each cell pack 1000a and/or b into intermediate weir 71 for storage, as detailed above. Advantageously, if production problems occur, the problematic line can be shut down, yet production of output solution can continue while the problem is rectified for the reasons provided above. If output solution is determined to be out of specification, it can be discarded to waste rather than stored. Once the output solution is produced according to the desired specifications, it can be sent to storage.

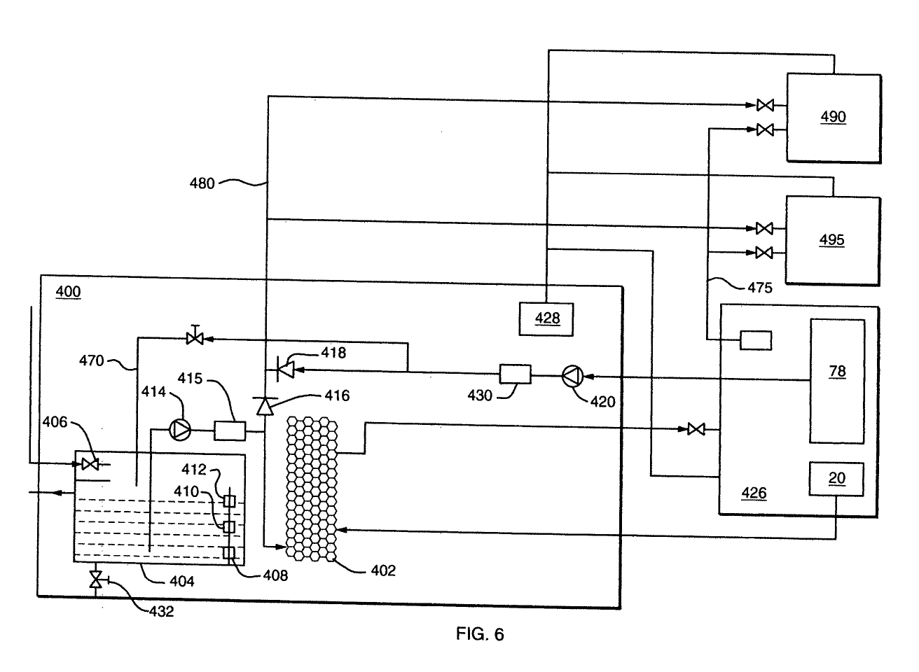

FIG. 6 is a flow diagram of an embodiment of the invention having additional water preparation apparatus 400 for preparing and storing water for the system. In some embodiments, water preparation apparatus 400 can replace combinations of other devices, such as a water softener, water storage tank, rinse water make-up tank, and control interface systems and equipment.

In some embodiments, water preparation apparatus 400 can store process water in process water tank 404. In some embodiments, process water tank 404 can receive potable water through an inlet. In some embodiments, the inlet may be controlled either manually or automatically by valve 406.

Process water tank 404 may contain water level sensors to detect the water level in tank 404. In some embodiments, level sensor 408 can detect a low level of process water in tank 404. If level sensor 408 detects a low process water level during normal production, an alarm may be sounded.

Level sensor 410 may detect a high water level in tank 404. In some embodiments, sensor 410 may signal to manually or automatically send water to the system. In some embodiments, sensor 410 may signal to manually or automatically enable pump 414 to send water to the system.

Level sensor 412 may detect an extra high process water level in tank 404. In some embodiments, an alarm may be sounded if level sensor 412 detects an extra high water level in tank 404. In some embodiments, inlet valve 406 may be automatically or manually closed upon detection of an extra high water level by sensor 412. In some embodiments, inlet valve 406 may be reopened if level sensor 412 detects the process water level has retreated below the extra high level. If level sensor 412 detects an extra high water level at a certain frequency which is predetermined to indicate a potential malfunction in the system, an alert may be sent to notify operators of a potential problem.

In certain embodiments, water tank 404 may hold up to about 60 liters of water. In some embodiments, water tank 404 may hold up to about 80 liters of water. In some embodiments, water tank 404 may hold up to about 120 liters. In some embodiments, water tank 404 can be a break tank. Water tank 404 can further include a class AA air break.

In some embodiments, the input of potable water into tank 404 is controlled by valve 406, which can be a solenoid valve. Valve 406 can control the filling of water tank 404. An isolation valve (not shown) may be positioned on the incoming water supply to tank 404. Tank 404 can further include drain valve 432.

In some embodiments, water can be dispensed from tank 404 by pump 414. In some embodiments, pump 414 can be centrifugal. Water dispensed from tank 404 can be delivered to an apparatus for producing an output solution having a predetermined level of available free chlorine which includes an electrolytic cell, means for passing a saline solution having a substantially constant chloride ion concentration through the cell, means for applying a substantially constant current across the cell, and means for dispensing output solution from the cell. In certain embodiments, water dispensed from tank 404 can be delivered to an apparatus as described above. In some embodiments, all or a portion of water dispensed by pump 414 can be delivered to water softener 402. In yet another embodiment, all or a portion of water dispensed from pump 414 can be provided to on or more devices in need of such water, e.g., endoscope cleaning devices 490 and 495 via line 480.

As noted above, certain embodiments can include water softener 402, and water softener 402 can receive water from water tank 404. In some embodiments, water softener 402 can contain resin bead bottles. For example, water softener 402 can contain one or more resin bead bottles. Each bottle can be 1-10 liters, more typically 1-8 liters, and more typically about 6 liters. In some embodiments, water softener 402 can supply at least 200 liters of softened water between regenerations. In some embodiments, water softener 402 can supply at least 400 liters of softened water between regenerations. In some embodiments, water softener 402 can supply at least 600 liters of softened water between regenerations.

In some embodiments, water softener 402 can provide about 50 to 400 liters per hour of softened water. In other embodiments, water softener 402 can provide about 100 to 300 liters per hour of softened water. In yet another embodiment, water softener 402 can supply softened water at a supply rate of about 10 to 30 liters per minute. In some embodiments, water softener 402 can supply softened water at a supply rate of about 15 to 25 liters per minute. In some embodiments, water softener 402 can supply softened water at a supply rate of about 20 liters per minute.

In some embodiments, water softener 402 can supply softened water with a hardness of about 0.5 to 5 ppm, having a hardness of 1 to 3 ppm and an alkalinity of up to about 200 mg/liter. In some embodiments, water softener 402 can supply softened water with an alkalinity of up to about 300 mg/liter.

In some embodiments, water softener 402 can supply softened water with a pH of 4.0-10.0. In other embodiments, the softened water can have a pH of 5.5-8.0. Typically, water softener 402 can supply softened water with a conductivity at 20° C. of up to 900 μS/cm. More typically, softened water can have a conductivity at 20° C. of up to 800 μS/cm.

In some embodiments, water softener 402 can include a control valve (not shown). In some embodiments, the control valve can be self-contained. In some embodiments, the control valve can control the water softener regeneration cycle.

In some embodiments, water softener 402 can receive brine. In some embodiments, water softener 402 can receive brine from tank 20. In some embodiments, brine can be used for water softener 402 regeneration.

In some embodiments, water preparation apparatus 400 can include a self-disinfection system, as described above. In some embodiments, the self disinfection system can include a means of providing output solution to tank 404 via line 470. Providing output solution to tank 404 can prevent microbial growth.

In some embodiments, water preparation apparatus 400 can produce rinse water. Rinse water may be produced using output solution by actuating valve 418, in combination with water from softener 402. In some embodiments, output solution can be provided from tank 78 via pump 420.

In some embodiments, rinse water can be produced by providing output solution to water flow line 480 which mixes with water dispensed from pump 414. In some embodiments, the output solution flow rate can be automatically or manually adjusted to match the water flow rate.

In some embodiments, the output solution flow rate can be monitored by flowmeter 430. In another embodiment, output solution can be directly provided to one or more devices in need of output solution, e.g., endoscope cleaning devices 490 and 495, via line 475.

In some embodiments, the rinse water can be stored in a tank. In some embodiments, pump 414 and flowmeter 415 provide water at a suitable rate, and pump 420 provides output solution. Flowmeter 430 can be monitored until the appropriate amount of output solution, based on the rinse water batch volume, is delivered.

In some embodiments, check valve 418 can prevent back flow of output solution into output solution storage tank 78. In some embodiments, check valve 414 can prevent back flow into process water storage tank 404. Similarly, check valve 416 can prevent back flow into water softener 402.

In some embodiments, water preparation apparatus 400 can have control interface equipment. The details of the control interface equipment for various embodiments are described in the following sections, and may be used with water preparation apparatus 400. The control interface equipment may control the timing and concentration of rinse water make-up, as the rinse water may be requested manually or automatically from throughout the system through the control interface. The production and delivery of softened water may be controlled by control interface equipment, and softened water may be automatically or manually requested through the control interface equipment. The control interface equipment may also control the valves by opening and closing the individual valves at the appropriate times based on the desired function of water preparation apparatus 400. For example, control unit 428 may communicate and interface with control unit 426 and with other devices, such as endoscope cleaning devices 490 and 495, which may require rinse water or output solution.

With respect to any embodiments described herein, as a further safety mechanism, it is highly desirable for the system producing the output solution to be self-monitoring. In this way, should any parameters, such as process or materials parameters, be detected to fall outside desired values, or any rapid or unexpected changes be detected, the system provide an alert and can then be adjusted as described further below. For example, measurements may indicate that more raw materials are required or that there is a fault in the production process. By incorporating self-monitoring in conjunction with an alert mechanism, the risk of generating a volume of output solution which is out of specification may be substantially reduced.

Advantageously, the system incorporates a self-alert mechanism which is preferably adapted to trigger a self-correction action and/or to notify a user of the system that there is a fault or demand. However, auto-correction, where possible, is preferred before an alarm is raised. For example, self-adjustment of flow rates may be all that is required to cope with fluctuations in local water pressure and alkalinity, whereas a disruption to the supply of input water may not necessarily be susceptible to auto-correction. As a yet further safety precaution or failsafe, it is preferred that production of output solution be stopped should self-correction not be possible or there be no response to an alarm. In this way, the possibility of dispensing output solution which fails to meet the desired parameters can be substantially avoided.

From another aspect, it is desirable if the system allows a user to interact with the production process, such as to obtain information on the performance of the system. Such interaction ideally allows the user to confirm that the production process is functioning properly and, if not, provides the user with guidance as to what action(s) can or should be taken to remedy any faults or deficiencies. Of course, any system faults or deficiencies which are not susceptible to auto-correction are likely to have been brought to a user's attention already by way of an alarm. In circumstances where faults or deficiencies are not easily remedied by the user, or where an indication is provided that the system will require servicing, the user may be prompted to call an expert.

However, it is useful to permit a user to interact with the system other than under alarm conditions, for instance to enable the user to ascertain whether or not there is sufficient output solution and/or bacteria-free rinse water to meet anticipated demand, to advise the user to wait for sufficient output solution to be generated, or to add salt and/or water. In addition, the user may be provided with information as to cell performance and/or its predicted lifespan thereby enabling the cell to be replaced at a convenient time, rather than having to react to a cell failure.

It will be appreciated that the user interface may be governed by computerized means, for example, with provision of suitable firmware and software. Typically, the system may be microprocessor controlled with the interface ideally provided through a display, keypad and/or printer means to provide on-site control.

While it is preferred that the process by which output solution is produced is self-adjusting, in the event that a fault cannot be rectified by self-adjustment, it is advantageous if self-diagnostic means are provided to identify where possible the nature of the fault.

Accordingly, it is preferred that the system of the invention further includes a service interface, through which an engineer may gain access to diagnostic information prior to taking remedial action. As with the user interface, the service interface will also be governed by suitable software.

For flexibility and convenience, it is preferred that service interface be accessed either on-site or remotely via a modem, wireless communication network, VPN, or the Internet. An advantage of permitting remote access is that an engineer may check the apparatus on a regular basis out having to travel to the site of the apparatus. This is of considerable benefit when the system has been installed in a far location.

The service interface may also be adapted to provide a history of the production process, for example how the production process has functioned over a period of time and hence to ascertain the remaining life expectancy of a particular component. Also the consumption of output solution can be monitored periodically. Different levels of access to the service interface may be provided, for example access to the production process history may be restricted to engineering personnel.

A further advantage is seen if a system engineer is provided with means to alter operating parameters remotely where possible, thus reducing the necessity for the engineer to attend the system if the process requires only minor adjustment. Also, this enables the engineer to monitor the system to keep it working smoothly. Indeed, by facilitating remote access, it is possible for an engineer to make adjustments to the system well before any alert mechanism is triggered. In such a way, intervention by the user can be kept to a minimum. Indeed, under typical conditions, a user may be required only to feed the system with salt at appropriate intervals, as any other controls or adjustments are made by the system itself or remotely through the service interface.

If remote access is provided via the Internet, for example, it may also include means by which the system can alert an engineer of a problem, for example, by e-mail, so that the apparatus may be attended to before a potentially more serious fault occurs. It is also possible to alert an engineer by fax, short message service (SMS) or other such means. All of these service interface features can help to reduce downtime of the apparatus and facilitate siting of apparatus in diverse locations.

Although the invention has been particularly described, it should be appreciated that the invention is not limited to the particular embodiments described and illustrated, but includes all modifications and variations falling within the scope of the invention as defined in the appended claims. For example, means other than the elongate, perforated dispenser described for mixing the concentrated salt solution with process water to produce a homogeneous saline solution may be used. Indeed, the concentrated salt solution can be continuously fed into a stream of process water rather than being pulse fed. In addition, while a weir tank is described as being particularly suitable for providing intermediate holding means for the output solution, other types of holding means may be used, such as a more conventional tank having appropriate outlet means for transferring its contents to the output solution storage tank, or a tube or pipe. The cell separator can be made of ceramics other than the aluminium oxide, zirconium oxide and yttrium oxide ceramic described and of any other suitable semi-permeable or ion-selective material.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims. Further, each and every reference recited herein is hereby incorporated in its entirety.

What is claimed:

1. An apparatus for producing an output solution having a predetermined level of available free chlorine comprising two or more parallel production lines, each production line comprising an electrolytic cell, means for passing a saline solution having a substantially constant chloride ion concentration through the cell, means for applying a current across the cell, and means for dispensing output solution from the cell.

2. The apparatus according to claim 1, wherein the electrolytic cells each comprise anode and cathode chambers separated by a separator, each chamber having a feed line through which the saline solution is fed into the chamber and anolyte and catholyte lines respectively for receiving the electrochemically treated solution.

3. The apparatus according to claim 2, wherein the output solution comprises the anolyte.

4. The apparatus according to claim 3, further comprising, for each production line, a catholyte recirculation line for feeding at least a portion of catholyte from the cathode chamber to the feed line of the anode chamber.

5. The apparatus according to claim 1, further comprising a concentrated salt solution make up tank, a process water tank and mixing means for mixing a concentrated salt solution from the make up tank with process water from the water tank to produce the saline solution.

6. The apparatus according to claim 5, wherein the mixing means comprises a dispenser for dispersing pulses of concentrated salt solution into a continuous flow of process water for each production line.

7. The apparatus according to claim 6, wherein the dispenser comprises a tube having a closed end, an open, feed end and a plurality of apertures along its length.

8. The apparatus according to claim 5, wherein the electrolytic cell of each production line is positioned at a level higher than the concentrated salt solution make up tank and the process water tank thereby to reduce back pressure on the cell.

9. The apparatus according to claim 1, further comprising an intermediate holding tank for receiving output solution from the cells.

10. The apparatus according to claim 9, further comprising measuring means to measure biocidal efficacy of the output solution in the intermediate holding tank.

11. The apparatus according to claim 10, wherein the measuring means comprises a pH meter and a redox probe.

12. The apparatus according to claim 9, further comprising a storage tank for receiving output solution from the intermediate holding tank.

13. The apparatus according to claim 12 wherein the intermediate holding tank comprises a weir tank located above the storage tank.

14. The apparatus according to claim 13, wherein the storage tank is positioned at a height to allow output solution to be dispensed therefrom by gravity feed.

15. The apparatus according to claim 9, further comprising a rinse water storage tank for receiving output solution from the intermediate holding tank and water.

16. The apparatus according to claims 15, wherein the rinse water storage tank is positioned at a height to allow rinse water comprising output solution diluted with water to be dispensed therefrom by gravity feed.

17. The apparatus according to claim 9, further comprising corrosion inhibitor storage and dispensing means for dosing corrosion inhibitor into the intermediate holding tank.

18. The apparatus according to claim 1, further comprising a user interface for displaying information on the performance of the apparatus, each production line, and the materials inputted to and outputted from the apparatus.

19. The apparatus according to claim 18, wherein the user interface includes a display with keypad controls.

20. The apparatus according to claim 18, further comprising control means to permit adjustment of operating parameters in response to information displayed.

21. The apparatus according to claim 1, further comprising a service interface for displaying diagnostic information on the performance of the apparatus and each production line.

22. The apparatus according to claim 21, wherein the service interface includes means to permit adjustment of operating parameters.

23. The apparatus according to claim 22 wherein the service interface includes means to permit adjustment of operating parameters specific to each production line.

24. The apparatus according to claim 21, wherein the service interface can be accessed remotely.

25. The apparatus according to claim 1, further including one or more failsafe mechanisms to prevent output solution from being dispensed when operating parameters cannot be adjusted to ensure that the solution has the required biocidal properties or when the output solution is older than a predetermined age.

26. A method of electrochemically treating a supply of aqueous salt solution in two or more parallel production lines, each line comprising an electrolytic cell having an anode chamber and a cathode chamber separated by a semi-permeable membrane, the anode and cathode chambers respectively being provided with an anode and a cathode, and each chamber having input and output lines for the solution being treated, wherein for each production line, i) aqueous salt solution is supplied to the anode and cathode chambers by way of their respective input lines, at least the cathode chamber input line being provided with a flow regulator, and output by way of their respective output lines; ii) a current is caused to flow between the anode and the cathode; and iii) a proportion of the solution output from the cathode chamber is recirculated to an input line of the anode chamber by way of a recirculation line.

27. The method according to claim 26, wherein for each production line the proportion of the solution output from the cathode chamber and recirculated to the input line of the anode chamber is determined by measuring the pH of the solution output from the anode chamber and using feedback control to maintain this pH at a substantially constant value.

28. The method according to claim 27, wherein the proportion of solution output from the cathode chamber and recirculated to the input line of the anode chamber is controlled by a pump on the recirculation line, the pump having a pump rate determined as a function of the measured pH of the solution output from the anode chamber.

29. The method according to claim 26, wherein the concentration of the aqueous salt solution is from 0.30 to 0.40% w/vol.

30. The method according to claim 27, wherein the pH of the solution output from the anode chamber for each production line is maintained at a value in the range of 6.0 to 7.0 inclusive.

31. The method according to claim 27, wherein for each production line gaseous products of electrolysis are removed from the solution output from the cathode chamber and recirculated to the input line of the anode chamber.

32. An apparatus for electrochemically treating a supply of aqueous salt solution, the apparatus comprising two or more parallel production lines each comprising an electrolytic cell having an anode chamber and a cathode chamber separated by a separator, the anode and cathode chambers respectively being provided with an anode or a cathode, and each chamber having input and output lines for the solution to be treated; wherein for each production line: i) the input line to the cathode chamber is provided with a flow regulator; ii) the anode and cathode are connected to a source of current; and iii) an output line from the cathode chamber is connected to an input line of the anode chamber by way of a recirculation line.

33. The apparatus as claimed in claim 32, wherein a pH probe is provided on the output line from the anode chamber for each production line.

34. The apparatus as claimed in claim 32, wherein a pump is provided on the recirculation line of each production line.

35. The apparatus as claimed in claim 34, wherein a pH probe, provided on the output line from the anode chamber for each production line, and the pump together form a feedback control mechanism for adjusting a flow rate of solution through the recirculation line so as to maintain a substantially constant pH of the solution output from the anode chamber.

36. An apparatus as claimed in claim 32, wherein a degassing unit is provided on the recirculation line of each production line.
